(19) United States
(12) Patent Application Publication
(10) Pub. No.: US 2024/0051908 A1
(43) Pub. Date: Feb. 15, 2024

(54) PROCESS FOR SEPARATING HEAVY BY-PRODUCTS AND CATALYST LIGAN FROM A VAPOUR STREAM COMPRISING ALDEHYDE

(71) Applicant: JOHNSON MATTHEY DAVY TECHNOLOGIES LIMITED, London (GB)

(72) Inventor: Martin SMIDT, London (GB)

(21) Appl. No.: 18/260,558

(22) PCT Filed: Feb. 23, 2022
(86) PCT No.: PCT/GB2022/050498

§ 371 Date: Jul. 6, 2023

(30) Foreign Application Priority Data
Feb. 25, 2021 (GB) 2102673.7

(51) Int. Cl. C07C 45/83; B01D 3/14; B01D 3/40

(52) U.S. Cl. CPC C07C 45/83; B01D 3/143; B01D 3/40

(57) ABSTRACT

A process for separating heavy by-products and catalyst ligand from a vapour stream comprising aldehyde, the heavy by-products and the catalyst ligand the process comprises passing the vapour stream to a fractionator in which the vapour stream is contacted with liquid aldehyde which removes at least a portion of the catalyst ligand and at least a portion of the heavy by-products from the vapour stream, recovering a liquid bottom stream, comprising removed catalyst ligand from the fractionator; recovering a scrubbed vapour stream from the fractionator, condensing a first portion of the scrubbed vapour stream to create the liquid aldehyde, and recovering a second portion of the scrubbed vapour stream as a product aldehyde stream. The liquid bottom stream is passed to a separation system to separate some aldehyde from the liquid bottom stream to create a recovered aldehyde stream, comprising the separated aldehyde.

### FIELD OF THE INVENTION

The present invention relates to a process for separating heavy by-products and catalyst ligand from a vapour stream comprising aldehyde. In particular, but not exclusively, the present invention relates to a process for separating heavy by-products and catalyst ligand from a vapour stream comprising aldehyde formed by passing a liquid output stream from a hydroformylation process, the liquid output stream comprising aldehyde, catalyst, catalyst ligand and heavy by-products, to a vaporiser and recovering the vapour stream from the vaporiser.

### BACKGROUND

The production of aldehyde by the hydroformylation of olefins is a well-known process. The aldehyde may be subjected to a variety of downstream reactions, including hydrogenation of the aldehyde to produce aliphatic alcohols, amination of the aldehyde to produce aliphatic amines, oxidation of the aldehyde to produce aliphatic acids, and aldol condensation reactions to produce acroleins, which are used, for example, in the production of plasticizers. Hydroformylation of olefins to aldehyde followed by hydrogenation of the aldehyde to produce aliphatic alcohols is a well-known use of the aldehyde. An example of such a process is the LP Oxo℠ process offered by Johnson Matthey and Dow. The hydroformylation is carried out in the liquid phase with a homogenous Rhodium catalyst modified with an organophosphorus ligand. Examples of such ligands and processes are disclosed in U.S. Pat. Nos. 4,148,830, 4,717,775 and 4,769,498. Organophosphines and organophosphites, particularly organomonophosphines, organobisphosphines, organotetraphosphines, organomonophosphites and organobisphosphites, are preferred organophosphorus ligands. The present invention may be of particular utility when the ligand has a vapour pressure of at least 0.01 mbar at 160° C. The present invention may be of particular utility when the ligand comprises triphenylphosphine (TPP) or triphenylphosphine oxide (TPPO), and especially when the ligand comprises TPP.

In a typical process, one or more hydroformylation reactors produce a product stream comprising the aldehyde and the homogenous catalyst. The aldehyde is separated from the catalyst by vaporisation, with the vaporised aldehyde leaving in the vapour phase and the catalyst liquor remaining as a liquid for recycling to the one or more hydroformylation reactors. The vaporisation is typically operated so as to prevent excessive carryover of the catalyst ligand in the vaporised aldehyde. However, it may be difficult to prevent the ligand being carried overhead in the vaporised aldehyde in levels that may be problematic in downstream hydrogenations. For example, ligand that is carried over may poison the catalyst used in the downstream hydrogenation. That may be particularly an issue in liquid phase hydrogenations; in vapour phase hydrogenations the vaporisation of the feed to the hydrogenation can potentially be used to remove carried over ligand. Even low levels of carried over ligand may poison catalysts. It may be that a ligand having a vapour pressure of 0.01 mbar or greater at 160° C. will result in unacceptable carry over.

Various solutions have been suggested for trying to mitigate ligand carryover. They include spraying a dispersed liquid, such as the product aldehyde, into the vaporised aldehyde stream to condense vaporised ligand, which can then be separated in a gas-liquid separator as described for example in U.S. Pat. No. 5,110,990. A problem with such arrangements is that heavy by-products in the aldehyde product vapour are also condensed by the dispersed liquid. Recycling the ligand thus results in an accumulation of the heavy by-products. It has been suggested that careful control of the dispersed liquid can promote the condensation of the ligand while avoiding the condensation of the heavy by-products, however in practice this is difficult to achieve and prone to problems. Any variations will result in either problematic carryover of the ligand through insufficient condensation of the ligand or accumulation of heavy by-products through over condensation of those by-products. Accumulation of heavy by-products may be an issue because the vaporiser may then need to be run at a higher temperature, which may lead to yet higher ligand carryover and so on. Using a purge from the catalyst recycle to remove the accumulated heavy by-products may lead to rhodium losses.

A further proposed solution is disclosed in US2018305285. In this system the vaporised aldehyde product stream is contacted with partial condenser so as to condense the phosphorous ligand and the by-products in the vaporised product aldehyde stream, wherein up to 10 weight percent of the vaporised aldehyde product stream is condensed. The condensed phosphorous ligand and the by-products are separated from the condensed aldehyde in a refining column, with the aldehyde being re-vaporised in the refining column and recycled to the vaporised aldehyde product stream. In some embodiments, the condensed phosphorous ligand and the by-products are not returned to the process, thus avoiding the accumulation of heavy by-products. In some embodiments, the phosphorous ligand can be separated and recycled from the by-product heavies in a separate distillation system. While such a system may avoid the accumulation of heavy by-products, the partial condenser provides only a single theoretical stage and thus there is a limit on the efficiency of ligand removal from the vaporised aldehyde product stream that can be achieved.

A further proposed solution is disclosed in U.S. Pat. No. 4,792,636. In that disclosure a process is provided for the recovery of an optionally substituted C₇ to C₁₇ aldehyde from a liquid hydroformylation product medium obtained by rhodium catalysed hydroformylation of an optionally substituted C₆ to C₁₆ olefin which contains (i) a rhodium complex hydroformylation catalyst containing rhodium in complex combination with carbon monoxide and with a ligand, (ii) excess ligand, (iii) at least one optionally substituted C₇ to C₁₇ aldehyde, and (iv) aldehyde condensation products, which process comprises:
(a) degassing said liquid hydroformylation medium;
(b) passing the degassed liquid hydroformylation medium through an evaporation zone maintained under temperature and pressure conditions conducive to evaporation of said at least one C₇ to C₁₇ aldehyde;
(c) recovering from the evaporation zone a liquid catalyst-containing stream;
(d) cooling the catalyst-containing stream exiting the evaporation zone;
(e) recovering a vaporous stream from the evaporation zone containing (i) at least one optionally substituted C₇ to C₁₇ aldehyde, (ii) ligand and (iii) a minor amount of said aldehyde condensation products;
(f) passing said vaporous stream to a fractionation zone;
(g) recovering from said fractionation zone (i) a vaporous product stream containing said at least one C₇ to C₁₇ aldehyde, and (ii) a liquid bottom stream containing said ligand and aldehyde condensation products;
and
(h) recycling said cooled catalyst-containing stream of step
(d) and at least a part of the material of said liquid bottom stream of step (g) to said hydroformylation zone.

At least a part of the liquid bottom stream can be recycled to the hydroformylation zone. Alternatively the bottom stream recovered from the fractionation zone can be passed to a ligand recover zone in which ligand is separated from aldehyde condensation products, for example by fractional distillation, and the resulting separated ligand can be recycled to the hydroformylation zone. It is also possible to recycle some of the bottom product stream from the fractionation zone to the hydroformylation zone and to treat the remainder in a ligand recover zone, from which recovered ligand is recycled.

In such a scheme any product aldehyde in the liquid bottom stream may be wasted. Attempts to reduce the level of product aldehyde in the liquid bottom stream may be made by running the bottom of the fractionation zone at a higher temperature, but such an approach may lead to excessive heavy by-product formation and even cracking in the reboiler of the fractionation zone. The heavy by-product formation undesirably consumes aldehyde that would otherwise be recovered as desired product. Any cracking products may contaminate the aldehyde product leaving the fractionation zone. The scheme may also not be practical for shorter chain aldehydes where the heavy by-products may become close boilers with the catalyst ligand.

In processes that include distillation columns to separate aldehyde isomers, such as a butanal isomer column to separate n-butanal from i-butanal, any carried over TPP can be removed in the isomer column. For example, the arrangements described in WO2017182780, would remove carried over TPP in the first, butanal isomer column, and thus the TPP would not be present in the feed sent on to the aldolization and subsequent hydrogenation where it may poison the catalyst. However, such an arrangement is unlikely to be economical when there is no commercial need to separate the isomers.

There therefore remains a need for a more efficient and effective system for preventing carryover of ligand in the vaporised product aldehyde stream. Preferred embodiments of the present invention seek to overcome one or more of the above disadvantages of the prior art. In particular, preferred embodiments of the present invention seek to provide improved processes for separating aldehyde from a hydroformylation product stream comprising the aldehyde, heavy by-products and catalyst ligand.

### SUMMARY OF INVENTION

According to a first aspect of the invention, there is provided a process for separating catalyst ligand from a vapour stream comprising aldehyde, heavy by-products and the catalyst ligand, the process comprising: passing the vapour stream to a fractionator in which the vapour stream is contacted with liquid aldehyde which removes at least a portion of the catalyst ligand and at least a portion of the heavy by-products from the vapour stream; recovering a liquid bottom stream, comprising removed catalyst ligand, removed heavy by-products and some of the aldehyde, from the fractionator; recovering a scrubbed vapour stream from the fractionator; condensing a first portion of the scrubbed vapour stream to create the liquid aldehyde, for reflux back to the fractionator; and recovering a second portion of the scrubbed vapour stream as a product aldehyde stream, wherein the liquid bottom stream is passed to a separation system to separate at least some of the aldehyde from the liquid bottom stream to create a recovered aldehyde stream, comprising separated aldehyde, and a waste stream comprising the removed catalyst ligand and the removed heavy by-products.

By passing the liquid bottom stream to the separation system to separate at least some of the aldehyde from the liquid bottom stream a more flexible system is created. For example, the system has the flexibility to operate the fractionator at conditions that reduce the potential for heavy by-product formation and potentially also cracking, while the separation system can be run at conditions to maximise aldehyde recovery in the recovered aldehyde stream. That may be advantageous because the fractionator is handling significantly larger flow rates and hence aldehyde inventory than the separation system and so heavy by-product formation and potentially cracking in the fractionator has the potential to create more significant problems. Heavy by-products may form in the reboiler for example by reactions involving the aldehyde. Such heavy by-product formation therefore represents a loss of the desired aldehyde product. Moreover, if cracking occurs, the cracking products may travel up the fractionator and contaminate the product aldehyde stream. The flexibility may also allow more economical performance because the larger fractionator may be run at more economical conditions, without the loss of aldehyde product that would occur in operating at those conditions in prior art processes.

The second portion of the scrubbed vapour stream may be condensed, preferably together with the first portion, and recovered as a liquid product aldehyde stream. The second portion of the scrubbed vapour stream may be recovered as a vapour stream, preferably by feeding the scrubbed vapour stream to a partial condenser, to condense the first portion, followed by a vapour-liquid separator to separate the first and second portions.

The process preferably comprises forming the vapour stream by passing a liquid output stream from a hydroformylation process, the liquid output stream comprising aldehyde, catalyst, catalyst ligand and heavy by-products, to a separator and recovering the vapour stream from the separator. The separator may, for example be a membrane separator, but is preferably a vaporiser. The vaporiser may, for example, comprise a heat exchanger and knock-out drum in series. The vapour stream preferably comprises 50-99 wt % of the aldehyde passed to the vaporiser. The vapour stream typically comprises a minor portion of the catalyst ligand and heavy by-products passed to the vaporiser. For example, the liquid output stream from the hydroformylation may comprise 5 wt % to 20 wt % catalyst ligand and the vapour stream preferably comprises not more than 5000 ppmw and preferably not more than 2500 ppmw catalyst ligand. As another example, up to 10 wt % of the catalyst ligand entering the vaporiser may be in the vapour stream. For short chain length olefins, such as C₃ or shorter, preferably not more than 1 wt % of the catalyst ligand entering the vaporiser is in the vapour stream. Preferably the catalyst and a majority of the catalyst ligand are recovered in a liquid stream in the vaporiser, typically at the bottom of the vaporiser, and are preferably recycled to the hydroformylation process.

By passing the vapour stream to a fractionator in which the vapour stream is contacted with liquid aldehyde, a more efficient separation of the ligand from the vapour stream may be achieved. That is because the fractionator provides the opportunity for multiple theoretical stages. Preferably the fractionator includes at least 2 theoretical stages, more preferably at least 4 theoretical stages. The theoretical stages may include a theoretical stage for a condenser associated with the fractionator. The use of liquid aldehyde condensed from the scrubbed vapour stream as reflux to the fractionator is an efficient source of scrubbing liquid that does not introduce further components into the system.

The liquid bottom stream is passed to a separation system, for example a distillation column, to separate at least some of the aldehyde from the liquid bottom stream to create a recovered aldehyde stream comprising the separated aldehyde and a waste stream comprising the removed catalyst ligand and the removed heavy by-products. Thus, the separated aldehyde, which is the valuable product of the hydroformylation, is not lost from the process. By not returning the removed heavy by-products to the process, the invention prevents the accumulation of heavy by-products in the process. The use of a fractionator advantageously creates the liquid bottom stream as a separate stream while also allowing an efficient, multi-theoretical stage separation to prevent excessive carryover of the catalyst ligand to downstream processes. Prior art processes that do not create a separate stream can lead to accumulation of the heavy by-products, while prior art processes with a single theoretical stage may not be as efficient at removing the catalyst ligand. Prior art processes in which aldehyde is not recovered from the liquid bottom stream either require operation at conditions that lead to undesirable aldehyde loss in the liquid bottom stream or conditions that minimise aldehyde in the liquid bottom stream but which can then lead to loss of aldehyde as a result of heavy by-product formation in the fractionator and potentially lead to cracking in the fractionator and contamination of the aldehyde product stream by cracking products. The process of the invention may also be operated at higher reflux ratios in the fractionator, thus increasing the proportion of the catalyst ligand and the heavy by-products removed from the vapour stream. Without the separation system, higher reflux ratios would either: require a fractionator reboiler operating at harsher conditions, e.g. increased temperature, to prevent higher levels of aldehyde in the liquid bottom stream; or would lead to higher levels of aldehyde loss.

The recovered aldehyde stream is preferably recycled to the process. The recovered aldehyde stream is preferably recycled upstream of the fractionator. The recovered aldehyde stream may be recycled to the fractionator. Preferably the vapour stream has been recovered from a vaporiser and the recovered aldehyde stream is preferably recycled to the vaporiser. Where the vaporiser comprises a knock-out drum the recovered aldehyde stream is preferably recycled to that knock-out drum. When the recovered aldehyde stream is recycled to the fractionator it is preferably recycled as a liquid reflux to the fractionator. In some embodiments the recovered aldehyde stream may be combined with the product aldehyde stream, however that may not be preferred as the recovered aldehyde stream may contain some catalyst ligand due to incomplete separation in the separation system.

Preferably the recovered aldehyde stream comprises at least 90 wt % and more preferably at least 95 wt % aldehyde. Higher aldehyde content in the recovered aldehyde stream may lead to increased possibilities for use of that stream. For example, if the aldehyde content is high, the desirability of combining the recovered aldehyde stream with the product aldehyde stream may be increased.

The waste stream preferably comprises not more than 10 wt %, and preferably not more than 7.5 wt % of the aldehyde. Thus, waste of the aldehyde, which is the desired product of the process, is kept low. The present invention advantageously achieves such low levels of the aldehyde in the waste stream, while also benefiting from a low bottom temperature in the fractionator. The waste stream is preferably sent to waste, thus ensuring that the removed heavy by-products are not returned to the process where they can accumulate.

When the separation system comprises a distillation column, the distillation column preferably includes a reboiler and a reflux condenser. It may still also be advantageous to have a reboiler on the fractionator. The separation system recovers aldehyde from the liquid bottom stream, and so even in the absence of a reboiler on the fractionator aldehyde losses are advantageously minimised. However, aldehyde recovered in the separation system will typically be recycled upstream, for example to an upstream hydrogenation reactor where the aldehyde will act as a diluent potentially meaning that the reactor needs to be larger to handle the extra flowrate. Providing a reboiler on the fractionator may advantageously provide flexibility to operate the reboiler in a manner, for example at a lower temperature, that allows more aldehyde to pass into the liquid bottom stream than would be desirable in a prior art arrangement, and which advantageously reduces risk of heavy by-products or cracking products being formed in the reboiler, but that nevertheless avoids excessive amounts of aldehyde being passed to the separation system and recycled. With a reboiler on the fractionator the invention may thus maintain the advantages of reduced heavy by-product formation and cracking, while benefiting from additional advantages of reduced dilution of the upstream process by recycled aldehyde. Having a reboiler on the fractionator may also allow an increase in the reflux ratio of the fractionator, thus increasing the proportion of the catalyst ligand and the heavy by-products removed from the vapour stream, while not increasing the amount of liquid aldehyde in the liquid bottom stream. Thus, greater removal of the catalyst ligand and by-product heavies may be achieved while maintaining the advantages of the invention set out above. The provision of the reboiler on the fractionator, in combination with the provision of the separation system, may also increase the flexibility of the process to handle changes, for example in the composition of input streams, by providing extra options for controlling the process. Provision of a reboiler may therefore enhance flexibility and ease of operability of the process.

The process comprises at least partially condensing the scrubbed vapour stream to create the liquid aldehyde, for reflux back to the fractionator. The process may comprise condensing the majority of the scrubbed vapour stream, for example by condensing essentially all of the aldehyde in the scrubbed vapour stream in a condenser, to create a condensed stream that is then split to create the liquid aldehyde for reflux to the fractionator and the product aldehyde stream. In that case the product aldehyde stream would be recovered as a liquid product aldehyde stream. The condensing may be carried out in a condenser, preferably followed by a knock-out drum or other vapour-liquid separator to remove any light components that have not condensed with the aldehyde. Alternatively, the process may comprise partially condensing the aldehyde in the scrubbed vapour stream and separating the condensed aldehyde, to create the liquid aldehyde for reflux back to the fractionator, from uncondensed aldehyde, which is recovered as the product aldehyde stream. The product aldehyde stream would therefore be a vapour product aldehyde stream. In such embodiments, the partial condensation is preferably carried out in a partial condenser, preferably followed by a knock-out drum or other vapour-liquid separator to separate the liquid aldehyde for reflux back to the fractionator from the vapour product aldehyde stream. The vapour product aldehyde stream may subsequently be condensed in a further condenser, preferably followed by a knock-out drum or other vapour-liquid separator to remove any light components that have not condensed with the aldehyde, to produce a liquid product aldehyde stream. Such an arrangement may be particularly attractive as a retro-fit to an existing plant and the invention may thus include a method of re-vamping a plant to install such an arrangement. The existing plant would have an existing condenser to condense the vapour stream and the existing condenser could be used as the further condenser, preferably without significant modification. The fractionator and partial condenser, and other equipment associated with the present invention, would be installed upstream of the existing condenser.

Preferably the vapour stream comprises at least 80 wt % aldehyde, more preferably at least 90 wt % aldehyde. It may be that the vapour stream comprises at least 0.5 wt % heavy by-products, or at least 1 wt % heavy by-products, or at least 2 wt % heavy by-products. Preferably the vapour stream comprises not more than 10 wt % heavy by-products, more preferably not more than 5 wt % heavy by-products. The invention may be particularly advantageous when the vapour stream comprises at least 10 ppmw catalyst ligand and even more so when the vapour stream comprises at least 20 ppmw catalyst ligand. If not removed, such levels of catalyst ligand may cause significant poisoning issues in downstream catalysts, particularly downstream hydrogenation catalysts. The vapour stream may comprise at least 100 ppmw or at least 200 ppmw catalyst ligand or at least 500 ppmw catalyst ligand. Preferably the vapour stream comprises not more than 5000 ppmw catalyst ligand and more preferably not more than 2500 ppmw catalyst ligand. The vapour stream may also comprise other contaminants, such as olefins, paraffins, alcohols and further contaminants, particularly light (i.e. lighter than the aldehyde) contaminants.

The product aldehyde stream preferably comprises not more than 10 ppmw catalyst ligand, more preferably not more than 5 ppmw and even more preferably not more than 1 ppmw catalyst ligand. Preferably at least 95 wt %, more preferably at least 97 wt %, yet more preferably at least 99 wt % and even more preferably at least 99.5 wt % of the catalyst ligand in the vapour stream is removed in the fractionator. Such high levels of catalyst ligand removal, and hence low levels of catalyst ligand in the product aldehyde stream may be possible in an economical manner because of the efficiency of removal in the fractionator.

The liquid bottom stream may comprise at least 5 wt % heavy by-products, or at least 10 wt % heavy by-products. The liquid bottom stream may comprise at least 60 wt % heavy by-products, and may comprise at least 80 wt % heavy by-products. However, advantageously, the invention may allow the fractionator to be operated in a manner such that more aldehyde enters the liquid bottom stream. Thus, the liquid bottom stream preferably comprises not more than 50 wt % heavy by-products. Preferably the liquid bottom stream comprises at least 25 wt %, and more preferably at least 50 wt % of the aldehyde. Higher aldehyde concentrations such as those typically result from milder conditions at the bottom of the fractionator, with the associated advantages of reduced heat duties and avoidance of undesirable heavy by-product formation and cracking reactions.

Preferably the reflux ratio of the fractionator is at least 0.05, and more preferably at least 0.1. The reflux ratio is the mass flowrate of the liquid aldehyde refluxed to the fractionator divided by the mass flowrate of the aldehyde product stream. The reflux ratio is preferably not more than 0.5. Higher reflux ratios, for example of at least 0.1, may be particularly advantageously used in the present invention because the separation system prevents excessive aldehyde loss.

When a reboiler is used on the fractionator the reboiler is preferably operated such that the temperature at the bottom of the fractionator is at least 90° C. The reboiler is preferably operated such that the temperature at the bottom of the fractionator is not more than 140° C. Operating at such temperatures may advantageously re-vaporise some of the aldehyde into the fractionator whilst avoiding the undesirable loss of aldehyde through formation of heavy by-products or formation of contaminants through cracking reactions.

The operating condition described above may be particularly advantageous when the catalyst ligand is TPP and the aldehyde comprises a C₃-C₆ aldehyde and particularly butyraldehyde.

Preferably the separation system comprises a distillation column. The distillation column is preferably operated such that the temperature at the bottom of the distillation column is greater than the temperature at the bottom of the fractionator. The distillation column is preferably operated such that the temperature at the bottom of the distillation column is not more than 140° C. The temperature at the bottom of the distillation column is preferably at least 90° C. and more preferably at least 100° C. The temperature at the bottom of the distillation column is preferably higher than the temperature at the bottom of the fractionator. The lower temperature at the bottom of the fractionator reduces the risk of heavy by-product formation or cracking in the fractionator, which handles a higher flow of material, while the higher temperature at the bottom of the distillation column increases recovery of the aldehyde into the recovered aldehyde stream. The temperature at the bottom of the distillation column is preferably at least 10° C., more preferably at least 20° C. and yet more preferably at least 30° C. higher than the temperature at the bottom of the fractionator.

The pressure of the distillation column is preferably at least 0.3 bara. The pressure of the distillation column is preferably not more than 1.2 bara. The pressure in the distillation column is preferably lower than the pressure of the fractionator. Preferably the pressure in the distillation column is at least 0.1 bar, preferably at least 0.2 bar and more preferably at least 0.5 bar lower than the pressure in the fractionator. Lower pressure typically means increased equipment size, but the distillation column handles lower flowrate than the fractionator and may thus still be a smaller piece of equipment. Lower pressure advantageously allows greater separation of the aldehyde into the recovered aldehyde stream without needing temperatures at which heavy by-product formation or cracking may occur. Thus, the fractionator may be operated at a higher pressure, so that the fractionator can be of an economical size, with some aldehyde allowed to slip into the liquid bottom stream, and the distillation column, which is handling a lower flowrate and thus is smaller anyway, operated at a lower pressure to achieve good recovery of the aldehyde without aldehyde losses to heavy by-product formation or contamination by cracking products.

The reflux ratio of the distillation column is preferably at least 0.1. The reflux ratio of the distillation column is preferably not more than 1.2. Such conditions, and particularly the combination of those temperature, pressure and reflux ratio ranges, may be particularly advantageous for recovering aldehyde in the recovered aldehyde stream while not losing aldehyde to heavy by-product formation or contaminating the recovered aldehyde stream with catalyst ligand or cracking products. Such conditions may be particularly suitable when the catalyst ligand is TPP and the aldehyde is a C₃-C₆ aldehyde.

The catalyst ligand preferably comprises an organophosphorus ligand. The organophosphorus ligand is preferably an organophosphine or organophosphite, particularly an organomonophosphine, organobisphosphine, organotetraphosphine, organomonophosphite or organobisphosphite. The present invention may be of particular utility when the ligand has a vapour pressure of at least 0.01 mbar at 160° C. The present invention may be of particular utility when the ligand comprises triphenylphosphine (TPP) or triphenylphosphine oxide (TPPO), and especially when the ligand comprises TPP.

The aldehyde is preferably a C₃ to C₆ aldehyde. More preferably the aldehyde comprises butyraldehyde or valeraldehyde, and most preferably the aldehyde comprises butyraldehyde.

In some embodiments the vapour stream may further comprise ligand decomposition products, which may themselves be organophosphorus compounds such as organomonophosphines, organobisphosphines, organotetraphosphines, organomonophosphites or organobisphosphites. The ligand decomposition products may be separated along with the catalyst ligand. Some ligand decomposition products may themselves act as catalyst ligands and the catalyst ligand may thus itself be a decomposition product of another catalyst ligand.

The skilled person will be familiar with the formation of heavy by-products in chemical processes to form aldehydes. The heavy by-products will typically include aldehyde condensation products, including aldehyde dimers and trimers.

The aldehyde condensation products may also include tetramers.

The skilled person will be aware of various fractionator designs, including packed beds and tray designs. The fractionator may be a scrubber.

The separation system preferably comprises a fractionator, such as a distillation column, but may for example be another type of separation system such as a membrane separation system.

Preferably the process comprises sending the aldehyde product stream to one or more reactors for hydrogenation of the aldehyde to produce an aliphatic alcohol, amination of the aldehyde to produce an aliphatic amine, oxidation of the aldehyde to produce an aliphatic acid, or aldol condensation reaction to produce an acrolein. Preferably the process comprises purifying the aliphatic alcohol, aliphatic amine, aliphatic acid, or acrolein, for example by distillation in one or more columns. The acrolein may be hydrogenated, preferably in a liquid phase hydrogenation, to an alcohol, preferably 2-ethylhexanol or 2-propylhexanol and most preferably 2-ethylhexanol, which is preferably then purified. Most preferably, the process comprises sending the aldehyde product stream to one or more reactors for hydrogenation of the aldehyde to produce an aliphatic alcohol, preferably butanol. The process preferably comprises purifying the alcohol, for example by distillation in one or more columns. The hydrogenation is preferably a liquid phase hydrogenation. The invention may be particularly beneficial when there is a downstream liquid hydrogenation, either of the aldehyde or of an acrolein produced by aldol condensation of the aldehyde, as catalysts for liquid hydrogenation may be particularly susceptible to poisoning by the catalyst ligand.

The process preferably comprises forming the vapour stream by passing a liquid output stream from a hydroformylation process, the liquid output stream comprising aldehyde, catalyst, catalyst ligand and heavy by-products, to a separator and recovering the vapour stream from the separator. The separator is preferably a vaporiser, which may, for example, comprise a heat exchanger and knock-out drum arranged in series. The hydroformylation process preferably comprises feeding catalyst, the catalyst ligand, olefins and carbon monoxide to one or more hydroformylation reactors; reacting the olefins with the carbon monoxide to form the aldehyde and the heavy by-products; and recovering the liquid output stream comprising the aldehyde, the catalyst, the catalyst ligand and the heavy by-products. The carbon monoxide is preferably comprised in syngas. Thus, the process preferably comprises feeding catalyst, catalyst ligand, olefins and carbon monoxide to one or more hydroformylation reactors; reacting the olefins with the carbon monoxide to form an aldehyde and heavy by-products; recovering a liquid output stream comprising the aldehyde, the catalyst, the catalyst ligand and the heavy by-products; passing the liquid output stream to a separator, recovering a vapour stream from the separator, the vapour stream comprising the aldehyde, preferably at least 50 wt % of the aldehyde, a minor portion of the catalyst ligand and a minor portion of the heavy by-product; passing the vapour stream to a fractionator in which the vapour stream is contacted with liquid aldehyde which removes at least a portion of the catalyst ligand and at least a portion of the heavy by-products from the vapour stream; recovering a liquid bottom stream, comprising removed catalyst ligand, removed heavy by-products and some of the aldehyde, from the fractionator; recovering a scrubbed vapour stream from the fractionator; condensing a first portion of the scrubbed vapour stream to create the liquid aldehyde, for reflux back to the fractionator; and recovering a second portion of the scrubbed vapour stream as a product aldehyde stream, wherein the liquid bottom stream is passed to a separation system to separate at least some aldehyde from the liquid bottom stream to create a recovered aldehyde stream, comprising the separated aldehyde, and a waste stream comprising the removed catalyst ligand and the removed heavy by-products.

Preferably the process comprises recovering a liquid stream from the separator, the liquid stream comprising the catalyst and a major portion of the catalyst ligand, and recycling the liquid stream to one or more of the hydroformylation reactors. The catalyst preferably comprises rhodium. When a stream is said to comprise a major portion of a component it may comprise at least 75 wt %, preferably at least 90 wt %, more preferably at least 95 wt % and even more preferably at least 99 wt % of the amount of that component fed to the separator and when a stream is said to comprise a minor portion of a component it may comprise not more than 25 wt %, more preferably not more than 10 wt %, more preferably not more than 5 wt % and even more preferably not more than 1 wt % of the amount of that component fed to the separator.

According to a second aspect of the invention, there is provided an aliphatic alcohol, aliphatic amine, aliphatic acid, or an acrolein obtained by a process according to the first aspect of the invention. Preferably the aliphatic alcohol, aliphatic amine, aliphatic acid, or acrolein is an aliphatic alcohol, most preferably butanol. Preferably, the aliphatic alcohol consists essentially of butanol. In another preferable aspect there is provided an aliphatic alcohol, preferably 2-ethylhexanol or 2-propylhexanol and most preferably 2-ethylhexanol, obtained by hydrogenating an acrolein obtained by a process according to the first aspect of the invention.

It will be appreciated that features described in relation to one aspect of the invention may be equally applicable in another aspect of the invention. Some features may not be applicable to, and may be excluded from, particular aspects of the invention.

### DESCRIPTION OF THE DRAWINGS

Embodiments of the present invention will now be described, by way of example, and not in any limitative sense, with reference to the accompanying drawings, of which:

FIG. 1 shows a comparative process;

FIG. 2 shows a comparative process;

FIG. 3 shows a comparative process;

FIG. 4 shows a comparative process;

FIG. 5 is a process according to the invention;

FIG. 6 is another process according to the invention;

FIG. 7 is another process according to the invention; and

FIG. 8 is another process according to the invention.

A comparative process is one for the purposes of comparing with the invention and may not be a prior art process.

### DETAILED DESCRIPTION

The following process examples were simulated using Aveva Simsci ProII. The skilled person will appreciate that the use of simulation packages is a well-established method for evaluating processes in the chemical field.

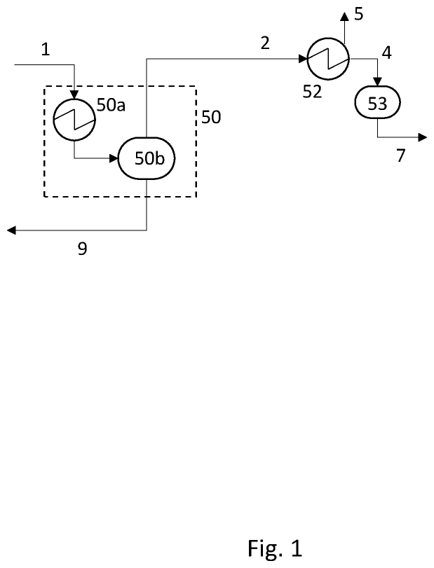

FIG. 1 shows a reference process which includes vaporiser 50, which comprises a heat exchanger 50a and a knock-out drum 50b, and condenser 52 but no fractionator. In this process, a Rh/TPP catalysed propylene hydroformylation using a liquid catalyst recycle scheme produces a flow containing 2.5 wt % propylene and propane, 70 wt % butyraldehyde, 15 wt % heavy by-products, 11.9 wt % TPP, with a balance of incondensable gases, catalyst and butanol. This feed 1 is fed to vaporiser 50 operating at 1.2 bara and 130° C., where approximately 70 wt % of the feed is vaporised, producing vapour stream 2 containing 3.5 wt % propylene and propane, 93.7 wt % butyraldehyde, 2.4 wt % heavy by-products, along with incondensable gases, butanol and about 1300 ppmw of TPP. Vapour stream 2 is fed to condenser 52 operating at 40° C., producing a liquid/vapour condenser outlet stream 4 which is passed to a knock-out drum 53, producing liquid aldehyde product stream 7 which contains approximately 1315 ppmw of TPP. Uncondensed vapours are vented in vent stream 5. A liquid stream 9 recycles the catalyst and TPP ligand to the hydroformylation.

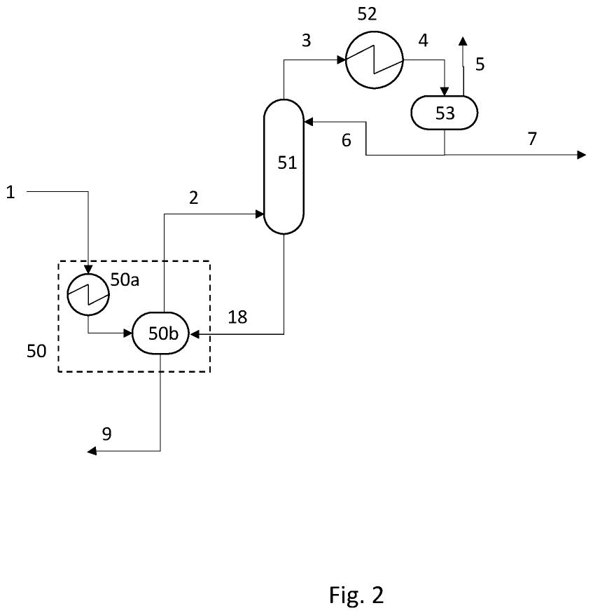

FIG. 2 shows a process as in FIG. 1 but further including fractionator 51. In FIG. 2, feed 1 (with the composition as described above) is fed to vaporiser 50, comprising heat exchanger 50a and knock-out drum 50b, producing vapour stream 2. Vapour stream 2 is fed to fractionator 51 which has four theoretical stages. The fractionator 51 operates at a pressure of 1.1 bara and has a bottom temperature of 78° C. Fractionator 51 receives a reflux of condensed overheads (reflux stream 6) to provide a reflux ratio of 0.3. Fractionator vapour stream 3 is fed to condenser 52 operating at 40° C., producing a liquid/vapour condenser outlet stream 4 which is passed to a knock-out drum 53. Uncondensed vapours are vented in vent stream 5 and remaining liquid is split into reflux stream 6 and liquid aldehyde product stream 7, which contains less than 1 ppb of TPP. In practice, this level of TPP is below detection limits. Fractionator liquid bottom stream 18 contains approximately 75 wt % butyraldehyde and is recycled to the knock-out drum 50b of the vaporiser 50. Substantially all the heavy by-products vaporised in vaporiser 50 will return via liquid bottom stream 18 and knock-out drum 50b to liquid stream 9 and return to the catalyst recycle. This will lead to further accumulation of heavy by-products in the catalyst solution, and hence further elevation of the temperature of vaporiser 50 will be required until the heavy by-products removal equals the heavy by-products formation. In this scenario, the heavy by-products cannot escape and may continue to accumulate until a purge is taken from the catalyst solution, for example from liquid stream 9. However, taking such a purge is undesirable because it can lead to loss of rhodium, which is expensive to replace.

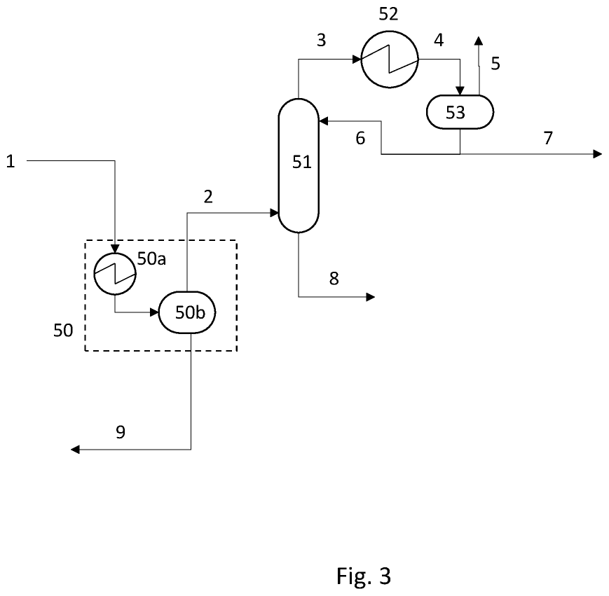

An alternative may therefore be as shown in FIG. 3. In that figure, where like numbered items are as in FIGS. 1 and 2 and not described again here, the liquid bottom stream 8, is sent to waste. In that way, heavy by-products do not accumulate. However, the entire liquid bottom stream 8, which as in FIG. 2 comprises approximately 75 wt % butyraldehyde, is sent to waste. The amount of butyraldehyde wasted may be quite substantial as the reflux ratio in fractionator 51 needs to be sufficiently large to keep the TPP level in aldehyde product stream 7 sufficiently low.

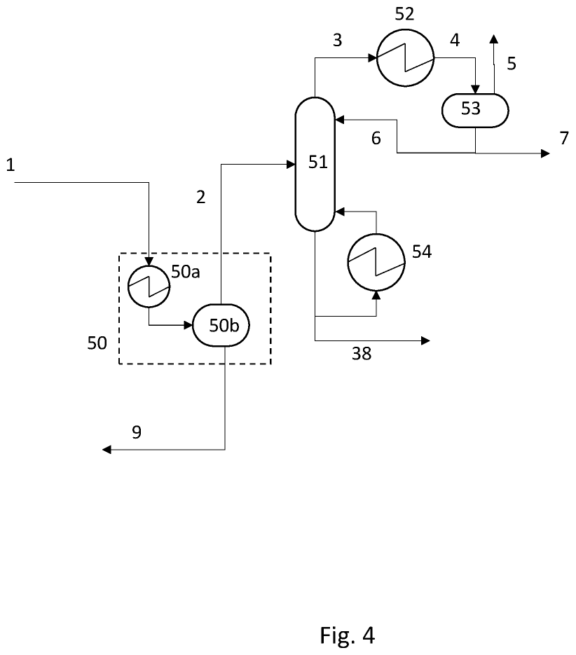

In FIG. 4, where like numbered items are as in FIGS. 1 to 3 and not described again here, the fractionator 51 includes a reboiler 54. The reboiler 54 concentrates the liquid bottom stream 38 by re-vaporising aldehyde and returning it to the fractionator 51. Fractionator 51 is operated at the same reflux ratio as described for FIGS. 2 and 3, and liquid aldehyde product stream 7 comprises less than 1 ppb TPP. Reboiler 54 is controlled to provide an aldehyde concentration of 5 wt % in liquid bottom stream 38, to minimize losses of aldehyde. Liquid bottom stream 38 is not recycled but purged as waste liquid. Reboiler 54 is operated at a temperature of 163° C. At such high reboiler temperature and low aldehyde concentration, the reboiler may be difficult to control, as the boiling point of the liquid will vary significantly with only small changes in the aldehyde concentration. This may result in unstable reboiler operation. Additionally, such high temperatures are likely to result in additional heavy by-product formation, resulting in loss of aldehyde, and possibly cracking of heavy by-products. Such cracking is undesirable as it can create light by-products, which are likely to contaminate the aldehyde product stream 7.

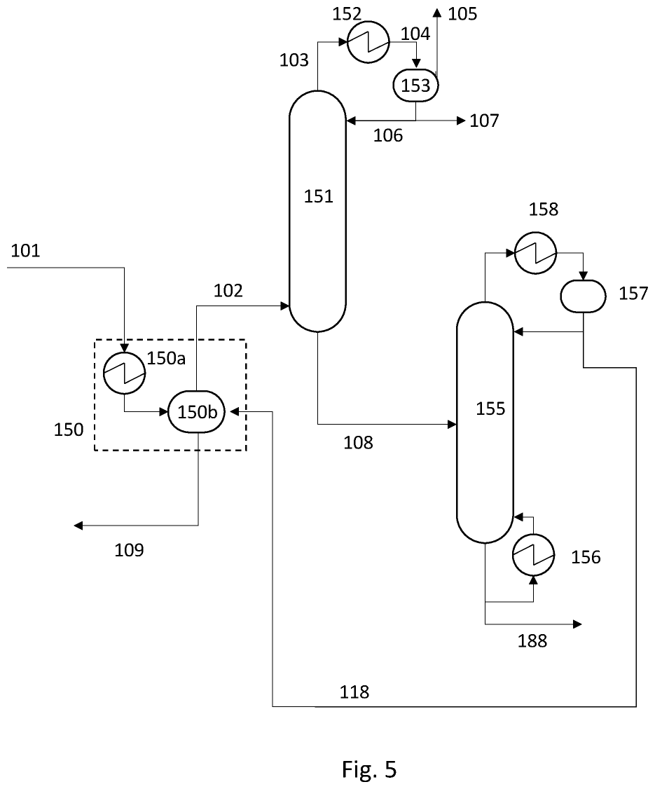

In FIG. 5, a process according to the invention includes a separation system 155 after the fractionator 151. Feed stream 101 having the same source and composition as feed stream 1 above is fed to a vaporiser 150, comprising heat exchanger 150a and knock-out drum 150b, to create vapour stream 102, which is passed to fractionator 151 as described above in relation to feed stream 1, vaporiser 50, vapour stream 2 and fractionator 51 of the previous figures. Fractionator 151 is operated at a reflux ratio of 0.3 and with a bottom temperature of 78° C. In a similar way to FIGS. 2 to 4, a scrubbed vapour stream 103 is recovered from the top of fractionator 151 and passed to condenser 152. Condenser 152 condenses most of the scrubbed vapour stream 103, including essentially all of the aldehyde in the scrubbed vapour stream 103, to produce a liquid/vapour condenser outlet stream 104, which is passed to a knock-out drum 153, from which uncondensed vapours are vented in a vent stream 105. The remaining liquid is split into a reflux stream 106 and a liquid aldehyde product stream 107. Liquid aldehyde stream 107 includes less than 1 ppb TPP. The liquid bottom stream 108 comprises 75 wt % butyraldehyde. The liquid bottom stream 108 is passed to a separation system, which in this embodiment comprises a further fractionator in the form of distillation column 155 operating at 0.5 bara, a reflux ratio of 0.1 and a temperature of 124° C. in the bottom of the distillation column 155. The distillation column 155 comprises a reboiler 156 and a condenser 158 and knock-out drum 157. A waste stream 188 is recovered from the bottom of the distillation column 155. The waste stream includes 5 wt % butyraldehyde. The waste stream 188 is sent to waste. From the top of the distillation column 155 a recovered aldehyde stream 118 is recovered, comprising butyraldehyde and possibly trace amounts of TPP and heavy by-products. In this embodiment the recovered aldehyde stream 118 is recycled to the knock-out drum 150b of the vaporiser 150. The recovered aldehyde stream 118 could also be recycled elsewhere in the process. In some embodiments the recovered aldehyde stream 118 may be combined with the product aldehyde stream 107.

The level of butyraldehyde in the waste stream 118 in this embodiment is the same as in the embodiment described above in relation to FIG. 4. However, it is achieved with a bottom temperature of 78° C. in fractionator 151 and a bottom temperature of 124° C. in distillation column 155, compared with a temperature of 163° C. in the reboiler 54 of fractionator 51 in FIG. 4. The lower temperatures may be advantageous, for example by reducing heat duties, and hence operating cost, by reducing aldehyde loss due to heavy by-product formation and/or by reducing contamination from cracking products. The process also has the advantage that the distillation column 155 is physically separated from the fractionator 151 and therefore can operate independently from the fractionator 151, unlike the reboiler 54 of FIG. 4. By feeding the liquid bottom stream 108 from the fractionator 151 into the distillation column 155, the distillation column 155 can operate almost independently from the fractionator 151, and hence any difficulty operating reboiler 156 on the distillation column 155 will not affect the operation of the fractionator 151. This may allow separation of difficult equipment from the main operation train and thereby facilitates ease of operation of the main train.

Results of the above examples are collected in the table below.

| | Units | FIG. 1 | FIG. 2 | FIG. 4 | FIG. 5 |
|---|---|---|---|---|---|
| TPP in aldehyde | ppmw | 1,315 | <1 ppb | <1 ppb | <1 ppb |
| product stream | | | | | |
| Heavy by-products in in | ppmw | 24,600 | <1 ppb | <1 ppb | <1 ppb |
| aldehyde product stream | | | | | |
| Reboiler duty for | MW | — | — | 0.57 | — |
| fractionator | | | | | |
| Reboiler duty for | MW | — | — | — | 0.42 |
| distillation column | | | | | |
| Fractionator bottom/ | ° C. | — | — | 163 | 78 |
| reboiler temperature | | | | | |
| Distillation column | ° C. | — | — | — | 124 |
| reboiler temperature | | | | | |

It is clear that the process described with respect to FIG. 5 is able to achieve an excellent aldehyde product stream 107 specification with reduced risk of aldehyde loss due to heavy by-product formation, reduced risk of contamination and increased operability, while requiring a lower duty versus the other processes.

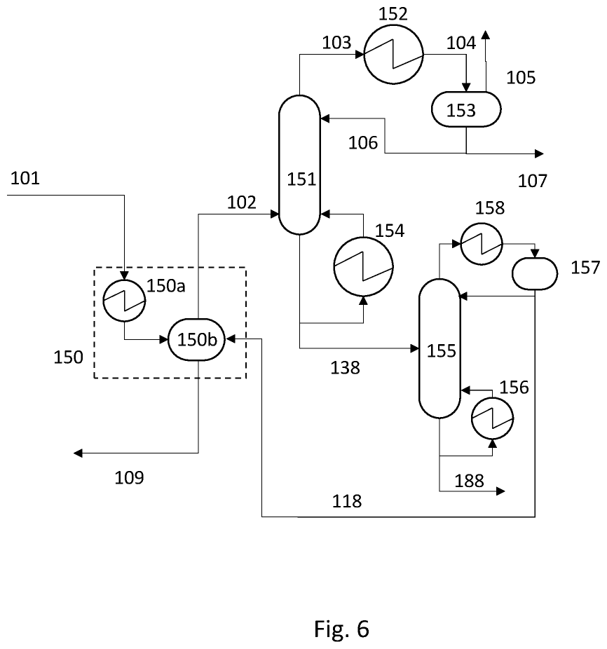

In FIG. 6, where like numbered items are as in previous figures and not described again here, the process is similar to that of FIG. 5, but this time including a reboiler 154 on the fractionator 151. The resulting liquid bottom stream 138 may thus have reduced aldehyde content compared to stream 108 in FIG. 5. Advantageously, the presence of reboiler 154 on fractionator 151 and reboiler 156 on distillation column 155 results in greater flexibility of operation. It may for example allow an optimised balance to be struck between keeping the bottom temperature of the fractionator 151 low enough to prevent heavy by-product formation and cracking reactions, but high enough to somewhat reduce aldehyde content in the liquid bottom stream 138. Although the distillation column 155 will recover aldehyde from the liquid bottom stream 138, the recovered aldehyde stream 118 is typically recycled to the knock-out drum 150b of the vaporiser 150, from where it is recycled to the hydroformylation reactor. That recycle prevents aldehyde loss, and so the advantage of the invention in preventing aldehyde loss in purges whilst also using temperatures that mitigates aldehyde loss to heavy by-product formation and contamination by cracking products is achieved, but recycling large amounts of aldehyde through an upstream hydroformylation reactor may undesirably dilute the reactants in the hydroformylation reactor. Having the reboiler 154 on fractionator 151 and the reboiler 156 on distillation column 155 may advantageously allow optimal mitigating of heavy by-product formation and contamination by cracking whilst avoiding excessive dilution by recycled aldehyde.

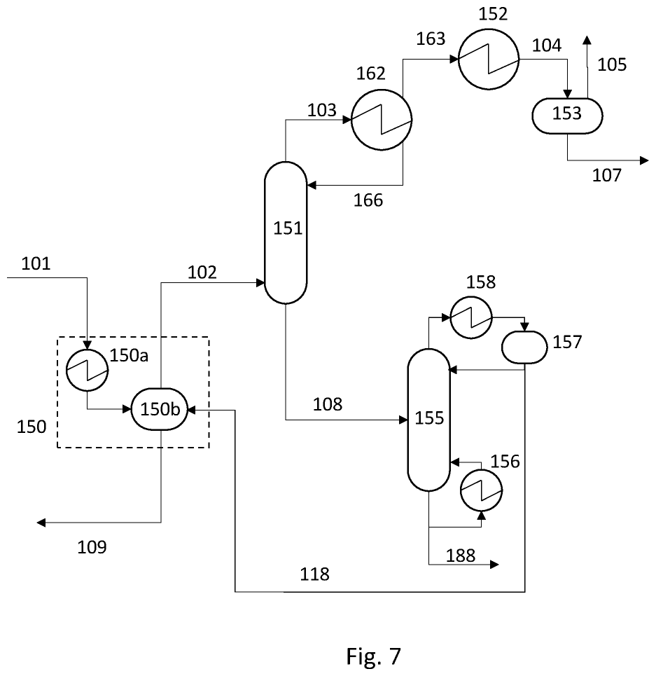

In FIG. 7, where like numbered items are as in previous figures and not described again here, the scrubbed vapour stream 103 is passed to a partial condenser 162. The partial condenser 162 condenses some of the aldehyde, which is returned to the fractionator 151 as reflux stream 166. The vapour outlet stream 163 from the partial condenser 162 is passed to a further condenser 152, where essentially all of the remaining aldehyde is condensed. The outlet 104 from the further condenser 152 is passed to knock-out drum 153, where it is separated into a vapour vent stream 105 and a liquid product aldehyde stream 107. Such an arrangement may be particularly attractive as a retrofit, as the plant will have an existing condenser to condense the vapour from the vaporiser 150, which can be used as the further condenser 152 without significant modification. The fractionator 151 and partial condenser 162 are then installed between the existing vaporiser 150 and further condenser 152. The partial condenser 162 depicted in FIG. 7 could equally be used in other embodiments of the invention, for example those described in relation to FIG. 5, 6 or 8.

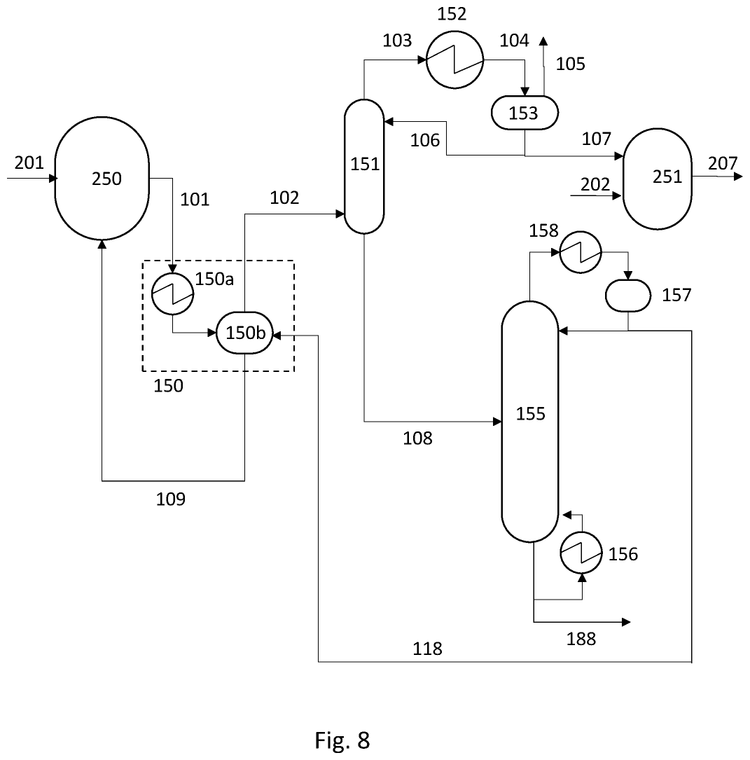

In FIG. 8, like numbered items are as in the process of previous figures and their description is not repeated. A stream 201 comprising propylene, carbon monoxide, rhodium and TPP is fed to a hydroformylation reactor 250. In the hydroformylation reactor 250, the propylene reacts with the carbon monoxide to form butyraldehyde, which exits the hydroformylation reactor 250 in liquid output stream 101, which is the feed stream to vaporiser 150, comprising heat exchanger 150a and knock-out drum 150b. From vaporiser 150 the vapour stream 102 and the liquid stream 109 are recovered. The liquid stream 109, comprising rhodium and TPP, is recycled to the hydroformylation reactor 250. The vapour stream 102 is processed in the fractionator 151 as described in relation to previous figures. The liquid product aldehyde stream 107 is sent to a hydrogenation reactor 251, along with a hydrogen-containing stream 202. In the hydrogenation reactor 251, the butyraldehyde from the liquid product aldehyde stream 107 reacts with hydrogen from the hydrogen stream 202 to form butanol, which is recovered in butanol product stream 207. The butanol product stream 207 may then be subject to purification steps, typically distillation, to recover a butanol product of a desired level of purity.

It will be appreciated by persons skilled in the art that the above embodiments have been described by way of example only, and not in any limitative sense, and that various alterations and modifications are possible without departure from the scope of the invention as defined by the appended claims. For example, the olefin may be butylene and the aldehyde may be valeraldehyde. As another example, the hydrogenation reactor 251 may be replaced with an amination reactor to produce an aliphatic amine, an oxidation reactor to produce an aliphatic acid, or an aldol condensation reaction to produce an acrolein, which may then be fed to a hydrogenation reactor to make an alcohol, with the hydrogen stream 202 being replaced with other reactant streams as will be apparent to the skilled person. While the vaporiser 150 comprises heat exchanger 150a and knock-out drum 150b, other designs of vaporiser could for example be used to create vapour stream 102.

1-20. (canceled)

21. A process for separating heavy by-products and catalyst ligand from a vapour stream comprising aldehyde, the heavy by-products and the catalyst ligand, the process comprising: passing the vapour stream to a fractionator in which the vapour stream is contacted with liquid aldehyde which removes at least a portion of the catalyst ligand and at least a portion of the heavy by-products from the vapour stream; recovering a liquid bottom stream, comprising removed catalyst ligand, removed heavy by-products and some of the aldehyde, from the fractionator; recovering a scrubbed vapour stream from the fractionator; condensing a first portion of the scrubbed vapour stream to create the liquid aldehyde, for reflux back to the fractionator; and recovering a second portion of the scrubbed vapour stream as a product aldehyde stream, wherein the liquid bottom stream is passed to a separation system to separate at least some aldehyde from the liquid bottom stream to create a recovered aldehyde stream, comprising the separated aldehyde, and a waste stream comprising the removed catalyst ligand and the removed heavy by-products.

22. The process according to claim 21 wherein the temperature at the bottom of the fractionator is not more than 140° C.

23. The process according to claim 21, wherein the separation system comprises a distillation column.

24. The process according to claim 23 wherein the temperature at the bottom of the distillation column is greater than the temperature at the bottom of the fractionator.

25. The process according to claim 23, wherein the pressure in the distillation column is lower than the pressure in the fractionator.

26. The process according to claim 23, wherein the distillation column is operated such that the temperature at the bottom of the distillation column is not more than 140° C.

27. The process according to claim 21, wherein the fractionator includes a reboiler.

28. The process according to claim 27, wherein the reboiler is operated such that the temperature at the bottom of the fractionator is at least 90° C.

29. The process according to claim 21, wherein the liquid bottom stream comprises at least 50 wt % aldehyde.

30. The process according to claim 21, wherein the recovered aldehyde stream is recycled to a point in the process upstream of the fractionator.

31. The process according to claim 21, wherein the catalyst ligand comprises an organophosphorus ligand.

32. The process according to claim 21, wherein the catalyst ligand comprises triphenylphosphine.

33. The process according to claim 21, wherein the catalyst ligand has a vapour pressure of at least 0.01 mbar at 160° C.

34. The process according to claim 21, wherein the aldehyde is a C₃ to C₆ aldehyde.

35. The process according to claim 21, wherein the process further comprises forming the vapour stream by passing a liquid output stream from a hydroformylation process, the liquid output stream comprising the aldehyde, catalyst, the catalyst ligand and the heavy by-products, to a separator and recovering the vapour stream from the separator.

36. The process according to claim 35, wherein the hydroformylation process comprises feeding catalyst, the catalyst ligand, olefins and carbon monoxide to one or more hydroformylation reactors; reacting the olefins with the carbon monoxide to form the aldehyde and the heavy by-products; and recovering the liquid output stream comprising the aldehyde, the catalyst, the catalyst ligand and the heavy by-products.

37. The process according to claim 21, wherein the process further comprises passing the aldehyde in the aldehyde product stream to one or more reactors for: hydrogenation of the aldehyde to produce an aliphatic alcohol; amination of the aldehyde to produce an aliphatic amine; oxidation of the aldehyde to produce an aliphatic acid; aldol condensation of the aldehyde to produce an acrolein; or aldol condensation of the aldehyde to produce an acrolein followed by hydrogenation of the acrolein to an aliphatic alcohol.

38. The process according to claim 37, wherein the process comprises passing the aldehyde in the aldehyde product stream to one or more reactors for: liquid phase hydrogenation of the aldehyde to produce an aliphatic alcohol; or aldol condensation of the aldehyde to produce an acrolein followed by liquid phase hydrogenation of the acrolein to an aliphatic alcohol.

39. The process according to claim 37, wherein the process comprises purifying the aliphatic alcohol, aliphatic amine, aliphatic acid, or acrolein.

40. An aliphatic alcohol, aliphatic amine, aliphatic acid, or an acrolein obtained by a process according to claim 37.
